(19) United States
(12) Patent Application Publication
(10) Pub. No.: US 2019/0374249 A1
(43) Pub. Date: Dec. 12, 2019

(54) ALIGNMENT ASSISTORS FOR TROCAR CANNULAS

(71) Applicant: Alcon Inc., Fribourg (CH)

(72) Inventors: Niels Alexander Abt, Winterthur (CH); Reto Grueebler, Schaffhausen (CH); Thomas Linsi, Shaffhausen (CH); Philipp Schaller, Stein am Rhein (CH)

(21) Appl. No.: 16/423,501

(22) Filed: May 28, 2019

Related U.S. Application Data
(60) Provisional application No. 62/681,377, filed on Jun. 6, 2018.

(51) Int. Cl. A61B 17/34; A61B 90/11; A61F 9/007

(52) U.S. Cl. CPC A61B 17/3423; A61F 9/007; A61B 90/11; A61B 17/3462

(57) ABSTRACT

Various embodiments are generally directed to alignment assistors, such as for guiding a surgical tool into axial alignment with a trocar cannula, for instance. Some embodiments are particularly directed to an alignment assistor that connects with a trocar cannula via a first end and receives and guides surgical instruments utilizing the trocar cannula into alignment with an axis of the trocar cannula. In one or more embodiments, for example, an apparatus for use in ocular surgery may include an alignment assistor with a coupling portion and a receiving portion joined via a base. In some embodiments, the coupling portion may include a trocar cannula coupler for connecting with a trocar cannula along an axis of the trocar cannula. The trocar cannula may provide a surgical instrument with access to an interior of an eye, along the axis, via the alignment assistor.

### PRIORITY CLAIM

This application claims the benefit of priority of U.S. Provisional patent application Ser. No. 62/681,377 titled “Alignment Assistors For Trocar Cannulas,” filed on Jun. 6, 2018, whose inventors are Niels Alexander Abt, Reto Grueebler, Thomas Linsi and Philipp Schaller, which is hereby incorporated by reference in its entirety as though fully and completely set forth herein.

### BACKGROUND

Generally, surgical instruments are tools or devices designed to perform specific actions involved in carrying out desired effects during surgery or operations. Sometimes a trocar cannula may be used to provide a surgical instrument with access to a surgical site, such as the interior of an eye. Typically, surgical instruments are used in ophthalmic surgery. Ophthalmic surgery typically includes performing an operation on an eye or its adnexa. Often ophthalmic surgeries utilize a probe. Further, these surgeries may include operations on the anterior portions of the eye as well as operations on the posterior portions of the eye. In various embodiments, ophthalmic surgery may be performed on a patient for therapeutic purposes.

### BRIEF DESCRIPTION OF THE DRAWINGS

FIGS. 1A and 1B illustrate an exemplary alignment assistor according to one or more embodiments described herein.

FIGS. 2A and 2B illustrate an exemplary process of coupling an alignment assistor with a trocar cannula according to one or more embodiments described herein.

FIGS. 3A-3D illustrate an exemplary process of inserting a surgical instrument tip through a capped trocar cannula assembly according to one or more embodiments described herein.

FIGS. 4A and 4B illustrate exemplary alignment assistors in conjunction with a trocar cannula and a surgical instrument tip according to one or more embodiments described herein.

FIGS. 5A and 5B illustrate an exemplary surgical instrument tip stiffener according to one or more embodiments described herein.

FIGS. 6A and 6B illustrate an exemplary process of inserting a surgical instrument tip with a surgical instrument tip stiffener through a trocar cannula according to one or more embodiments described herein.

FIG. 7 illustrates an exemplary surgical instrument tip stiffener in conjunction with a surgical instrument tip and a trocar cannula according to one or more embodiments described herein.

### DETAILED DESCRIPTION

Various embodiments are generally directed to alignment assistors, such as for guiding a surgical tool into axial alignment with a trocar cannula, for instance. Some embodiments are particularly directed to an alignment assistor that connects with a trocar cannula via a first end and receives and guides surgical instruments utilizing the trocar cannula into alignment with an axis of the trocar cannula. In one or more embodiments, for example, an apparatus for use in ocular surgery may include an alignment assistor with a coupling portion and a receiving portion joined via a base. In many embodiments, the coupling portion may include a trocar cannula coupler for connecting with a trocar cannula along an axis of the trocar cannula. In many such embodiments, the trocar cannula may provide a surgical instrument with access to an interior of an eye, along the axis, via the alignment assistor. In various embodiments, the receiving portion may include a guidance port. In various such embodiments, the guidance port may be shaped to guide alignment of the surgical instrument when the surgical instrument utilizes the trocar cannula to access the interior of the eye via the guidance port.

Some challenges facing trocar cannulas include difficult and time-consuming procedures to insert a surgical instrument into a trocar cannula without inhibiting and/or damaging the surgical instrument. The challenges may result from an inability to guide proper alignment of a surgical instrument with a trocar cannula. For instance, the surgical instrument may include a soft or flexible tip that can become kinked when inserted into a trocar cannula with improper alignment. In some such instances, if the surgical instrument continues to be inserted into the trocar cannula without removing the kink, then surgical complications may occur, including damage to the trocar cannula, damage to the surgical instrument, and injury to the patient. In various embodiments, this may cause a surgeon to go through multiple attempts before successfully inserting a surgical instrument into a trocar cannula. These and other factors may result in unreliable trocar cannulas with limited flexibility, deficient performance, and safety concerns. Such limitations can reduce the capabilities, usability, and applicability of the trocar cannula, contributing to inefficient devices with limited abilities.

Various embodiments described herein include an alignment assistor that couples with a trocar cannula and promotes alignment of a surgical instrument with an axis of the trocar cannula via a guidance port. For instance, the guidance port may include a funnel shape to guide alignment of a surgical instrument with the axis of the trocar cannula to access a surgical site, such as the interior of an eye. In some instances, the guidance port may include a membrane with a concave surface shaped to guide axial alignment of a surgical instrument with the trocar cannula. In some such instances, the opening may comprise one or more slits in a membrane. In these and other ways one or more of the alignment assistors described herein may function in a safe and efficient manner to achieve better performing trocar cannulas, resulting in several technical effects and advantages.

Reference is now made to the drawings, wherein like reference numerals are used to refer to like elements throughout. In the following description, for purpose of explanation, numerous specific details are set forth in order to provide a thorough understanding thereof. It may be evident, however, that the novel embodiments can be practiced without these specific details. In other instances, well-known structures and devices are shown in block diagram form in order to facilitate a description thereof. The intention is to cover all modification, equivalents, and alternatives within the scope of the claims.

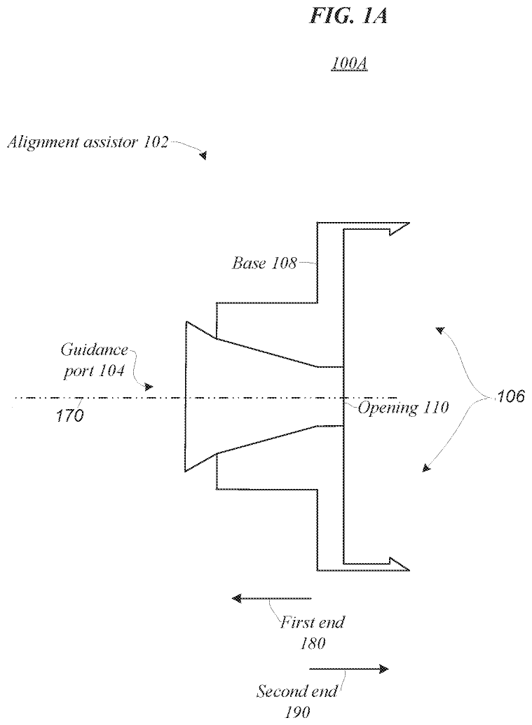

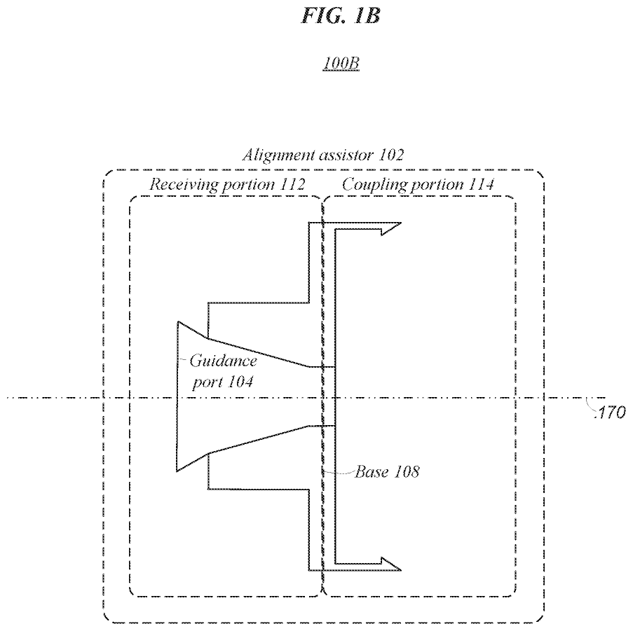

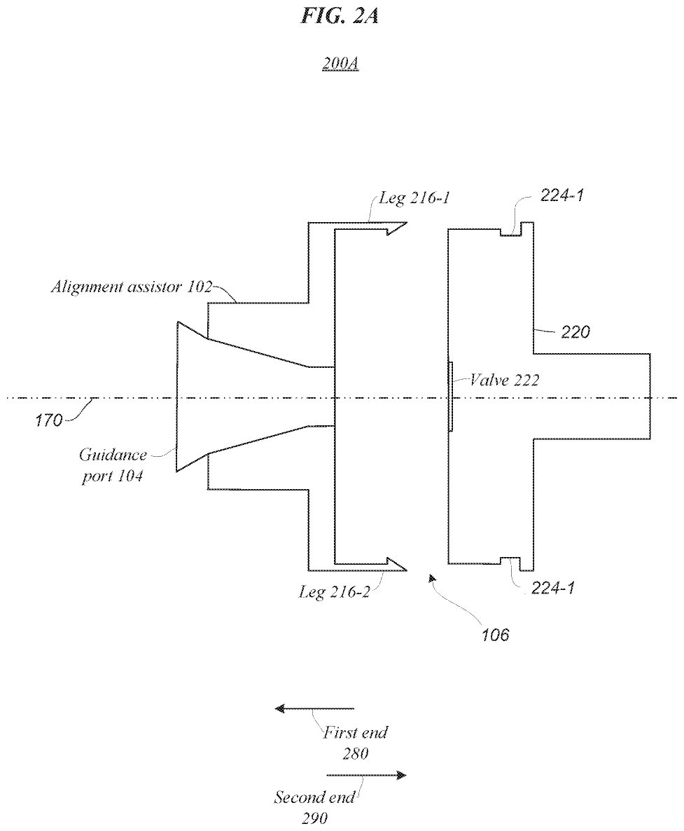

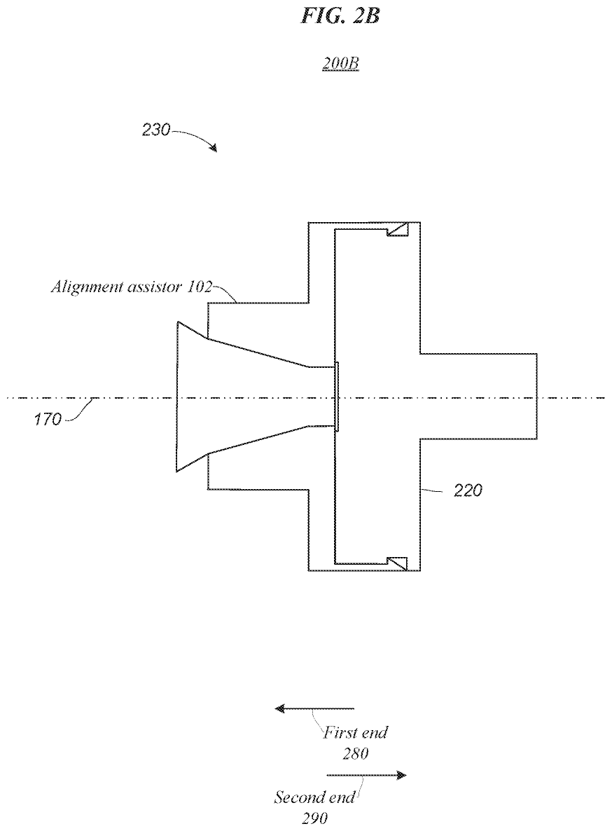

FIGS. 1A and 1B illustrate an embodiment of an operating environment 100 that may be representative of various embodiments. Operating environment 100 may include an alignment assistor 102. In one or more embodiments described herein, alignment assistor 102 may be utilized to guide alignment of a surgical instrument with an entry axis 170 such that the surgical instrument can be inserted through a trocar cannula (see e.g., FIG. 2A) for access to a surgical site. In various embodiments, alignment assistor 102 may include a guidance port 104, a trocar cannula coupler 106, and a base 108. As shown in FIG. 1B, base 108 may connect a receiving portion 112 that includes guidance port 104 to a coupling portion 114 that includes trocar cannula coupler 106. In some embodiments, trocar cannula coupler 106 of coupling portion 114 may be used to attach alignment assistor 102 with a trocar cannula along entry axis 170. In one or more embodiments described herein, guidance port 104 may be shaped to guide a surgical instrument inserted in the receiving portion 112, proximate first end or proximal end 180, such that it aligns with entry axis 170 prior to exiting opening 110, passing base 108, and exiting second end or distal end 190. Embodiments are not limited in this context.

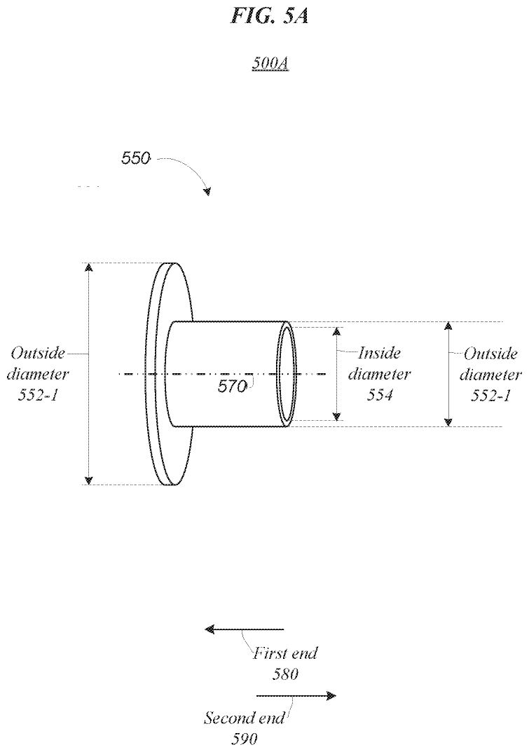

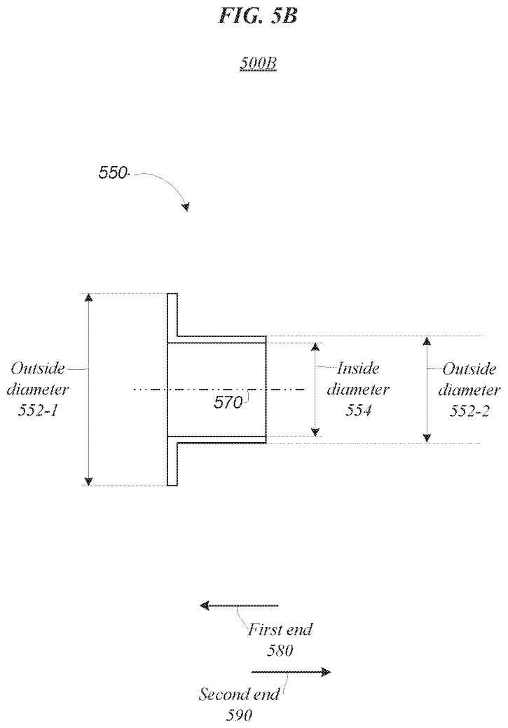

In prior situations in which a surgical instrument is inserted through a trocar cannula, such as a standard valved trocar cannula, if the surgical instrument is not properly aligned with the axis of the trocar cannula, the misalignment could lead to kinks and/or damage to the surgical instrument. For example, a soft tipped surgical instrument may become kinked or blocked by the trocar cannula, such as at a valve in a valved trocar cannula. In one or more embodiments described herein, alignment assistor 102 may be coupled to a trocar cannula such that an axis of the trocar cannula aligns with entry axis 170. In one or more such embodiments, a surgical instrument inserted into the guidance port 104 of receiving portion 112 may be guided into alignment with entry axis 170. In some embodiments, guidance port 104 may align a surgical instrument with entry axis 170 such that a surgical instrument with a soft or flexible tip, such as a backflush or soft tip cannula, can be passed through alignment assistor 102 and a trocar cannula attached thereto without becoming kinked, tangled and/or blocked. In various embodiments, one or more additional or alternative components, such as a surgical instrument tip stiffener (see e.g., FIGS. 5A and 5B), may be coupled to the surgical instrument to prevent the surgical instrument from becoming kinked, tangled and/or blocked.

In one or more embodiments, trocar cannula coupler 106 of coupling portion 114 may attach to a trocar cannula such that entry axis 170 aligns with an axis of the trocar cannula. In the illustrated embodiment, trocar cannula coupler 106 is located proximate the second or distal end 190 of alignment assistor 102. In various embodiments, trocar cannula coupler 106 may couple and uncouple from one or more trocar cannulas one or more times. In some embodiments, trocar cannula coupler 106 may utilize or include a semi-rigid material, such as a polymer, to connect with the trocar cannula along entry axis 170. As will be discussed in more detail below, in various embodiments, one or more portions of the trocar cannula coupler 106 may engage with corresponding portions of the trocar cannula to connect with the trocar cannula along the entry axis 170.

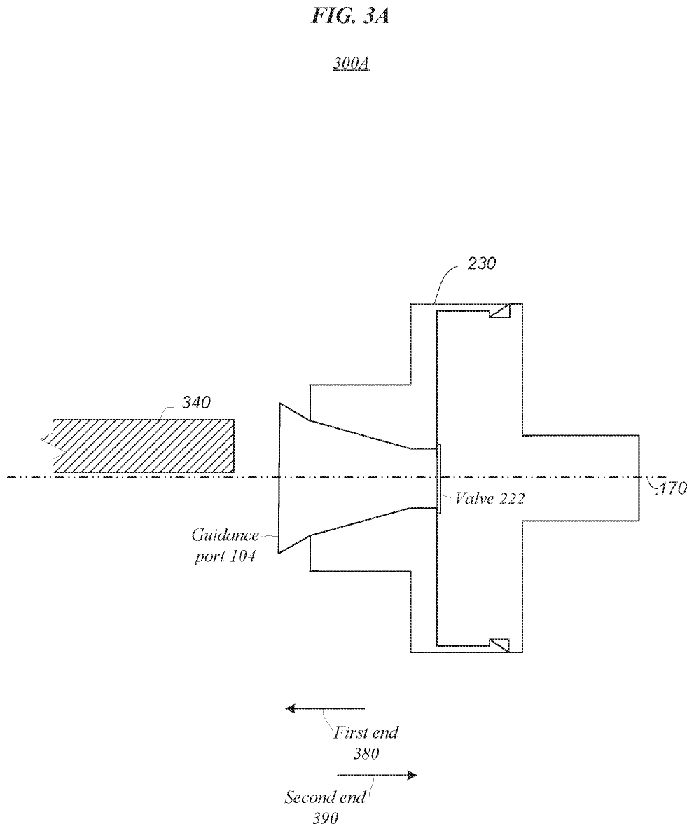

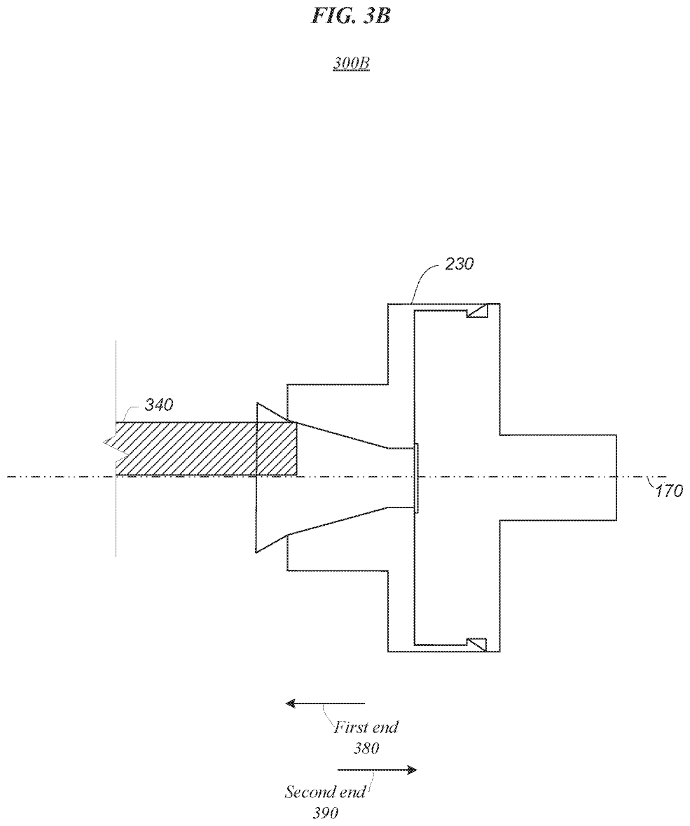

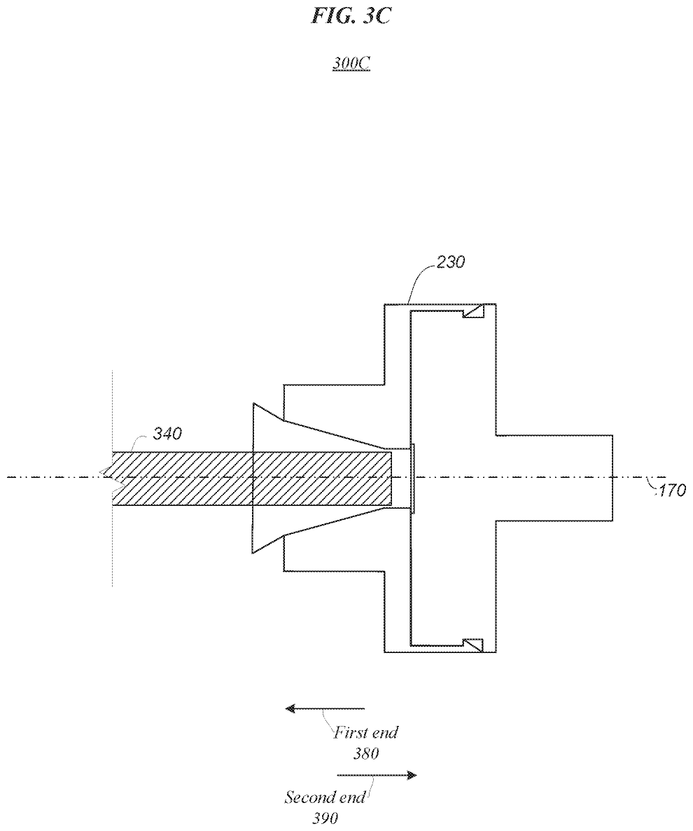

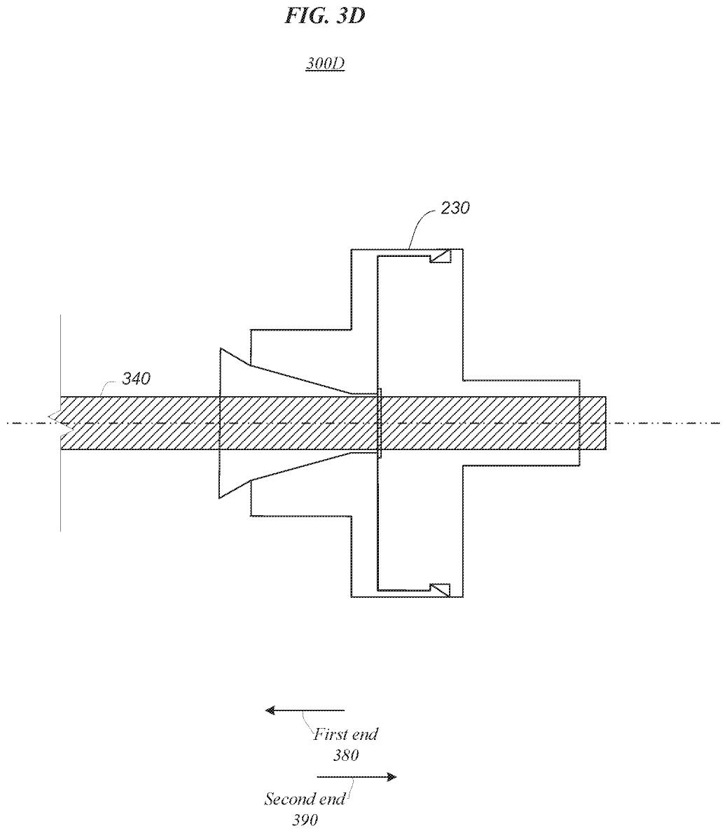

In one or more embodiments, guidance port 104 of receiving portion 112 may align a surgical instrument with entry axis 170 such that a surgical instrument with a soft or flexible tip can be passed through alignment assistor 102 without becoming kinked, tangled and/or blocked. In the illustrated embodiment, guidance port 104 is located proximate the first end 180 of alignment assistor 102. As previously mentioned, in various embodiments described herein, alignment assistor 102 may include guidance port 104 that is shaped to promote alignment of a surgical instrument tip 340 (e.g., a cannula) with entry axis 170 of a trocar cannula. For instance, the guidance port 104 may include a funnel shape to guide alignment of surgical instrument tip 340 (FIGS. 3A-3D) with the entry axis 170 of the alignment assistor 102 to access a surgical site. For example, the guidance port 104 may comprise a tapered surface that decreases in diameter as a distance to the distal end 190 of the guidance port decreases to guide alignment of the surgical instrument with the entry axis 170. In some instances, the guidance port 104 may include a membrane with opening 110. In some such instances, the membrane may have a concave surface shaped to guide axial alignment of a surgical instrument with a trocar cannula attached to trocar cannula coupler 106. In various instances, the concave surface may be exposed to an ambient pressure. In some embodiments, guidance port 104 may utilize an elastic material to conform with surgical instruments passed therethrough. In various embodiments, the guidance port 104 may be constructed from silicon or latex. Other materials are also contemplated.

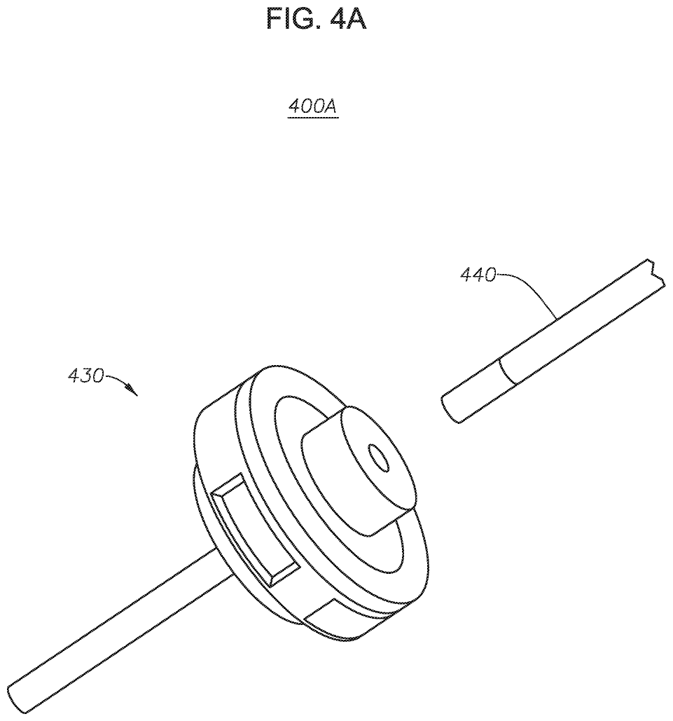

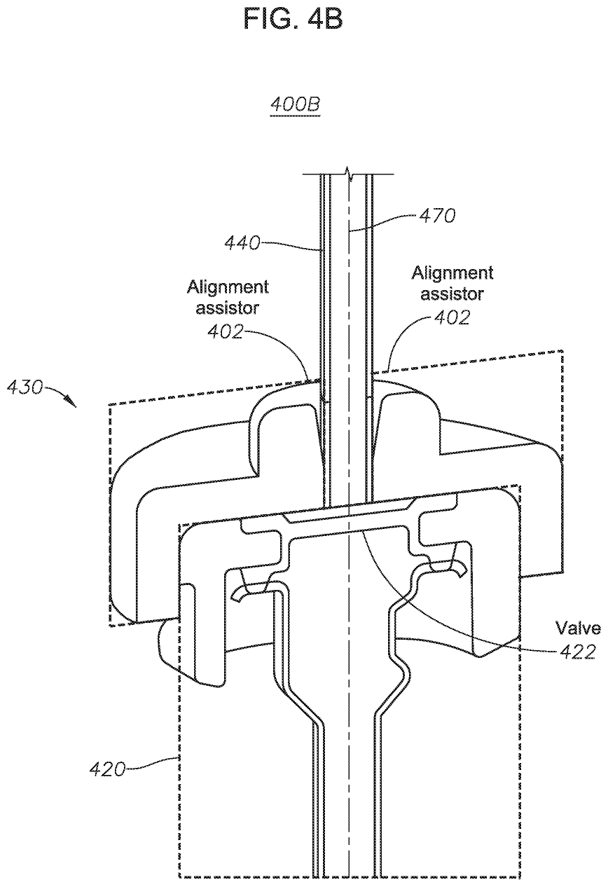

In many embodiments, insertion of a surgical instrument tip, such as a soft tip cannula, through alignment assistor 102 may be simplified and expedited with guidance port 104. In some embodiments, guidance port 104 may include a membrane with a cylindrical or tapered hollow aligned with entry axis 170 (see e.g., FIG. 4). In some such embodiments, a diameter of the hollow may decrease when moving towards opening 110. In many such embodiments, when a surgical instrument is inserted in the hollow proximate first end 180, the hollow provides guidance such that it can be inserted past base 108 and through a trocar cannula valve (e.g., valve 222) without becoming kinked or damaged. In one or more embodiments, opening 110 may include one or more slits, holes, or ports a surgical instrument may pass through. For example, opening 110 may include a cylindrical hole aligned with entry axis 170. In one or more embodiments, the inner diameter of the opening 110 may match the inner diameter of a trocar cannula attached thereto (see e.g., FIG. 2A).

In various embodiments, guidance port 104 may include a narrowing structure with a larger diameter at the first or proximal end 180 narrowing to a smaller diameter at the second or distal end 190, such as a funnel or a tapered or conical hole. For instance, a surgical instrument may be inserted in the narrowing structure of alignment assistor 102, and the narrowing structure may act as guidance as the surgical instrument passes through opening 110 and past base 108. In these and other ways alignment assistor 102 may prevent bending or kinking of surgical instruments utilizing alignment assistor 102 for access to a surgical site, such as the interior of an eye.

FIGS. 2A-2B illustrate an embodiment of a process that may be representative of various embodiments. In various embodiments, the process may include attaching a trocar cannula 220 with valve 222 to alignment assistor 102 along entry axis 170. In the illustrated embodiments, the process may include first and second states 200A, 200B of alignment assistor 102 in conjunction with trocar cannula 220. In the first state 200A, alignment assistor 102 and trocar cannula 220 are aligned along entry axis 170 with the second end 190 of alignment assistor 102 facing the first end 280 of trocar cannula 220. In the second state 200B, alignment assistor 102 and trocar cannula 220 may be connected with trocar cannula coupler 106 with at least a portion of the second end 190 of alignment assistor 102 contacting the first end 280 of trocar cannula 220. In some embodiments, the alignment assistor 102 and trocar cannula 220 coupled together in the second state 200B may be referred to as a capped trocar cannula assembly 230. In one or more embodiments described herein, a surgical instrument inserted into the guidance port 104 of capped trocar cannula assembly 230 may be guided into alignment with entry axis 170 such that the surgical instrument passes through valve 222 and out of the second end 290 of the capped trocar cannula assembly 230 without becoming kinked or damaged. Embodiments are not limited in this context.

In one or more embodiments, trocar cannula coupler 106 of alignment assistor 102 may attach to trocar cannula 220 along entry axis 170. In some embodiments, trocar cannula coupler 106 may couple and uncouple from one or more trocar cannulas one or more times. In various embodiments, one or more portions of the trocar cannula coupler 106 (e.g., legs 216-1, 216-2) may engage with corresponding portions of the trocar cannula 220 to connect with the trocar cannula 220 along the entry axis 170. In the illustrated embodiment, the corresponding portions of the trocar cannula 220 that engage with trocar cannula coupler 106 can include notches 224-1, 224-2 to receive the triangular protrusions on each of legs 216-1, 216-2.

In many embodiments, trocar cannula coupler 106 may utilize or include a semi-rigid material, such as a polymer, to connect with the trocar cannula 220 along entry axis 170. In some embodiments, legs 216-1, 216-2 may be constructed from a semi-rigid material to enable trocar cannula coupler 106 to securely attach to trocar cannula 220. For instance, when transitioning from first state 200A to second state 200B, legs 216-1, 216-2 may flex outward and away from entry axis 170 and then inward such that at least a portion of the triangular portions of the legs 216-1, 216-2 are located in notches 224-1, 224-2, respectively. Similarly, in some instances, legs 216-1, 216-2 may flex outward and away from notches 224-1, 224-2 to remove alignment assistor 102 from trocar cannula 220. Various other mechanisms may be used for coupling the alignment assistor to the trocar cannula, including but not limited to snap fit, friction fit, screw threads, clasps, adhesive, and the like.

FIGS. 3A-3D illustrate an embodiment of a process that may be representative of various embodiments. In various embodiments, the process may include insertion of a surgical instrument tip 340 (e.g., a cannula) through capped trocar cannula assembly 230. In the illustrated embodiments, the process may include first, second, third, and fourth states 300A, 300B, 300C, 300D of capped trocar cannula assembly 230 in conjunction with surgical instrument tip 340. In the first state 300A, surgical instrument tip 340 is misaligned with entry axis 170 of capped trocar cannula assembly 230. In the second state 300B, surgical instrument tip 340 is inserted into capped trocar cannula assembly 230 while still misaligned with entry axis 170. In one or more embodiments described herein, due to being misaligned with entry axis 170, surgical instrument tip 340 may contact a portion of guidance port 104 that guides the surgical instrument tip 340 toward alignment with entry axis 170. In the third state 300C, insertion has continued such that guidance port 104 has guided surgical instrument tip 340 into axial alignment with capped trocar cannula assembly 230 along entry axis 170. In the fourth state 300D, surgical instrument tip 340 has been inserted through capped trocar cannula assembly 230 such that surgical instrument tip 340 extends along entry axis 170 past the second end 390 of capped trocar cannula assembly 230. Embodiments are not limited in this context.

In one or more embodiments, guidance port 104 may align a surgical instrument with entry axis 170 such that a surgical instrument with a soft or flexible tip 340 (e.g., a cannula) can be passed through the capped trocar cannula assembly 230 without becoming kinked, tangled and/or blocked. For example, a soft tip cannula (e.g., surgical instrument tip 340) may become kinked or blocked at valve 222 if the surgical instrument tip 340 is misaligned with entry axis 170 when it passes through valve 222. As previously mentioned, in various embodiments described herein, alignment assistor 102 may include guidance port 104 that is shaped to promote alignment of a surgical instrument tip 340 (e.g., a cannula) with entry axis 170. For instance, the guidance port 104 may include a funnel shape to guide alignment of surgical instrument tip 340 with the entry axis 170 to access a surgical site, such as the interior of an eye. In some instances, the guidance port 104 may include a concave surface shaped to guide axial alignment of surgical instrument tip 340 with trocar cannula 104. In some embodiments, surgical instrument tip 340 may include a soft or flexible tip.

In some embodiments, surgical instrument tip 340 may need to extend past the second end 390 of capped trocar cannula assembly by a predetermined gauge length. In some such embodiments, alignment assistor 102 of capped trocar cannula assembly 230 may include a deformable portion to allow surgical instrument tip 340 to extend past the second end 390 of capped trocar cannula assembly 230 by the predetermined gauge length. For instance, guidance port 104 may compress along entry axis 170 to allow surgical instrument tip 340 to extend past the second end 390 of capped trocar cannula assembly 230 by the predetermined gauge length. In such instance, guidance port 104 may be constructed from a flexible material that returns to its original shape after flexing.

FIGS. 4A and 4B illustrate operating environments 400A, 400B, which may be representative of various embodiments. In one or more embodiments, operating environment 400A includes a perspective view of the capped trocar cannula assembly 430 in conjunction with surgical instrument tip 440. In various embodiments, operating environment 400B includes a cross section of capped trocar cannula assembly 430 in conjunction with surgical instrument tip 440. In many embodiments, capped trocar cannula assembly 430 may be the same or similar to capped trocar cannula assembly 230. In operating environment 400A, surgical instrument tip 440 may be proximate capped trocar cannula assembly 430. For instance, operating environment 400A may be in the same or similar state as illustrated in FIG. 3A. In operating environment 400B, surgical instrument tip 440 may be inserted into alignment assistor 402 such that it is aligned with valve 422 and trocar cannula 420 along entry axis 470, however surgical instrument tip 440 has not passed through valve 422. For example, operating environment 400B may be in the same or similar state as illustrated in FIG. 3C. Embodiments are not limited in this context.

FIGS. 5A and 5B illustrate operating environments 500A, 500B, which may be representative of various embodiments. Operating environment 500A may include a perspective view of surgical instrument tip stiffener 550 with first and second ends 580, 590. Operating environment 500B may include a cross-section view of surgical instrument tip stiffener 550 with first and second ends 580, 590. In one or more embodiments described herein, surgical instrument tip stiffener 550 may stiffen (e.g., structurally support) a flexible surgical instrument inserted therethrough. For instance, surgical instrument tip stiffener 550 may enable surgical instrument tip 340 to be inserted through the valve of a trocar cannula without becoming kinked or damaged. In the illustrated embodiments, surgical instrument tip stiffener 550 may include a tubular structure along entry axis 570 with outside diameters 552-1, 552-2, inside diameter 554, and entry axis 570. Embodiments are not limited in this context.

In various embodiments, the cylindrical structure of surgical instrument tip stiffener 550 may be rigid. In some embodiments, the cylindrical structure of surgical instrument tip stiffener 550 may include a rigid portion and a flexible portion. For example, the rigid portion may be proximate the second end 590 (e.g., distal end) of surgical instrument tip stiffener 550 and the flexible portion may be proximate the first end 580 (e.g., proximal end) of surgical instrument tip stiffener 550. In many embodiments, the surgical instrument tip stiffener 550 may include a tube. For instance, the surgical instrument tip stiffener 550 may include a tube that can be slid over a soft portion of a surgical instrument tip (e.g., with a soft tip cannula). In such instances, the surgical instrument tip stiffener 550 may keep the surgical instrument tip straight during insertion through a valve of a trocar cannula, preventing kinking or damage to the surgical instrument tip. In one or more such embodiments, the tube may transform into a cone with inside diameter 554 at the second end 590. In some embodiments, one or more portions of surgical instrument tip stiffener 550 may be attached to a trocar cannula or a capped trocar cannula assembly.

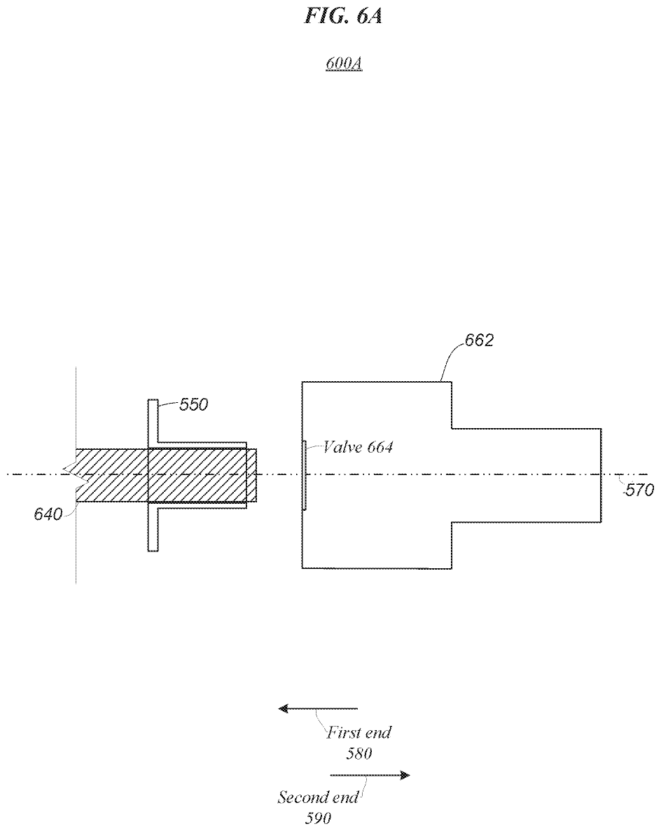

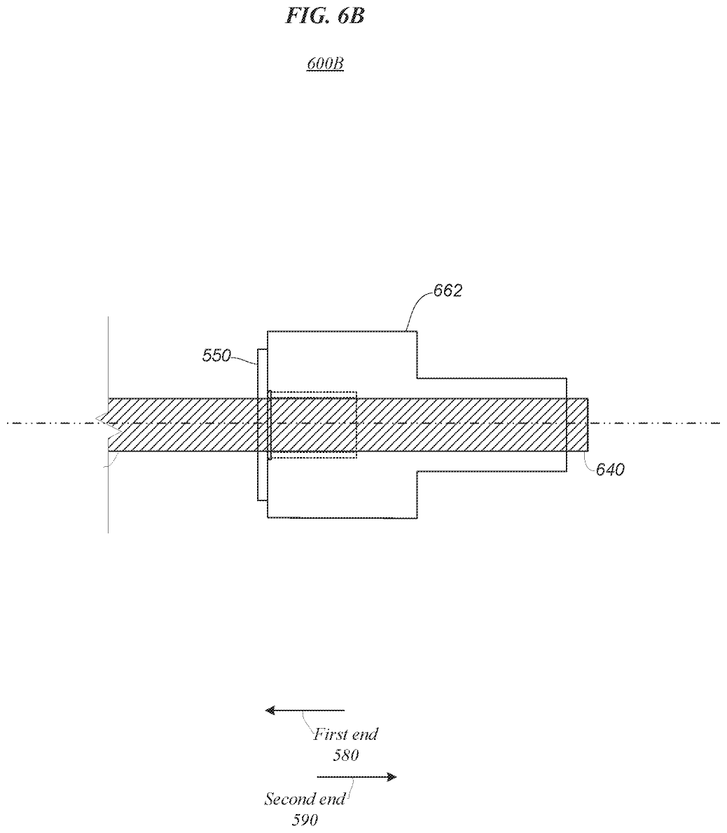

In one or more embodiments, the inside diameter 554 may extend from the first end 580 to the second end 590 of surgical instrument tip stiffener 550. In one or more such embodiments, this may enable at least a portion of a surgical instrument tip, such as a soft tip cannula, to pass, along entry axis 570, from the proximal end to the distal end of surgical instrument tip stiffener 550. In other words, and as will be described in more detail below (see e.g., FIGS. 6A and 6B), a surgical instrument may be inserted into the first end 580 of surgical instrument tip stiffener 550 such that is exits the second end 590 of surgical instrument tip stiffener 550. In some embodiments, inside diameter 554 may remain constant between the first and second ends 580, 590 of surgical instrument tip stiffener 550. In other embodiments, inside diameter 552 may vary between the first and second ends 580, 590 of surgical instrument tip stiffener 550. For example, a first inside diameter proximate the first end 580 may gradually decrease to a second inside diameter proximate the second end 590.

In various embodiments, outside diameter 552-1 and outside diameter 552-2 may be different. For instance, outside diameter 552-1 may be larger than outside diameter 552-2. As shown in the illustrated embodiments, surgical instrument tip stiffener 550 may include a stepped transition from outside diameter 552-1 to outside diameter 552-2. For instance, outside diameter 552-1 may comprise a portion of a flange. In other embodiments, surgical instrument tip stiffener 550 may include a sloped transition from outside diameter 552-1 to outside diameter 552-2. For example, outside diameter 552-1 may gradually transition into outside diameter 552-2 between the first and second ends 580, 590 of surgical instrument tip stiffener 550. In many embodiments described herein, surgical instrument tip stiffener 550 may be used in conjunction with one or more of a surgical instrument tip 340 (e.g., a cannula), a trocar cannula (e.g., trocar cannula 220), and a capped trocar cannula assembly (e.g., capped trocar cannula assembly 230). In some such embodiments, the trocar cannula may include a valved trocar cannula.

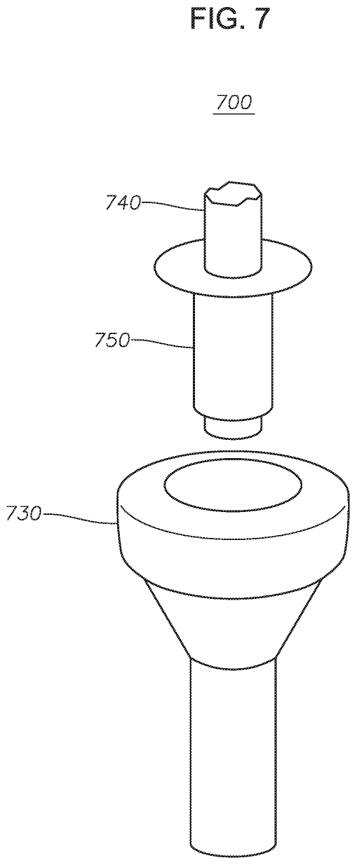

FIGS. 6A and 6B illustrate an embodiment of a process that may be representative of various embodiments. In various embodiments, the process may include inserting surgical instrument tip 640 through trocar cannula 662 along entry axis 570. In one or more embodiments described herein, surgical instrument tip 640 may utilize a surgical instrument tip stiffener 550 to prevent the surgical instrument tip 640 from kinking or becoming damaged when trying to pass through valve 664. In some embodiments, trocar cannula 662 may be the same or similar to trocar cannula 220. In various embodiments, trocar cannula 662 may be replaced with a capped trocar cannula assembly (e.g., capped trocar cannula assembly 230). In other words, in one or more embodiments, surgical instrument tip stiffener 550 may be used in conjunction with a capped trocar cannula assembly (see e.g., FIG. 7). Embodiments are not limited in this context.

As previously mentioned, FIGS. 6A and 6B may illustrate a process that may include inserting surgical instrument tip 640 through trocar cannula 662 along entry axis 570. In one or more embodiments, an outside diameter of surgical instrument tip 640 may be less than or equal to the inside diameter 554. In the illustrated embodiments, the process may include first and second states 600A, 600B of surgical instrument tip stiffener 550 in conjunction with surgical instrument tip 640 and trocar cannula 662. In the first state 600A, surgical instrument tip stiffener 550 may be disposed over surgical instrument tip 640. For example, surgical instrument tip stiffener 550 may be slid over surgical instrument tip 640. In some embodiments, surgical instrument tip stiffener 550 may include or utilize a tube that slides over surgical instrument tip 640. In some such embodiments, a first end 580 of the tube may have a conical shape that has the inner diameter of the surgical instrument tip stiffener 550. In some embodiments, the surgical instrument tip stiffener 550 may include a stiffening sleeve and/or tube. In various embodiments, surgical instrument tip 640, surgical instrument tip stiffener 550, trocar cannula 662, and valve 664 may be aligned with entry axis 570 in first state 600A. In some instances, surgical instrument tip stiffener 550 may include a conical shape on the exterior to guide surgical instrument tip 640 into alignment with trocar cannula 662 along entry axis 570.

In the second state 600B, the second end 590 of surgical instrument tip 640 may extend past the second end of trocar cannula 662. In an illustrated embodiment, surgical instrument tip stiffener 550 may be stopped at the first end 580 of trocar cannula 662 such that only a portion of surgical instrument tip stiffener 550 extends past valve 664 when surgical instrument tip 640 is inserted through trocar cannula 662. In such embodiments, the outside diameter 552-2 may be less than or equal to an inside diameter of valve 664. In other embodiments, no portion of surgical instrument tip stiffener 550 may extend past valve 664 when surgical instrument tip 640 is inserted through trocar cannula 662. In other such embodiments, outside diameter 552-2 may be greater than or equal to the inside diameter of valve 664. In some embodiments, the surgical instrument tip stiffener 550 may be removable once surgical instrument tip 640 is inserted through valve 664. For instance, to utilize the entire gauge length of the surgical instrument tip (e.g., distance surgical instrument tip 640 extends past second end 590 of trocar cannula 662), the surgical instrument tip stiffener 550 may be removed. In some embodiments, surgical instrument tip stiffener 550 may deform to enable the entire gauge length to be utilized.

FIG. 7 illustrates an embodiment of an operating environment 700 that may be representative of various embodiments. Operating environment 700 may include a surgical instrument tip stiffener 750 in conjunction with surgical instrument tip 740 and capped trocar cannula assembly 730. In one or more embodiments described herein, surgical instrument tip stiffener 750 and/or capped trocar cannula assembly 730 may be utilized to guide alignment of a surgical instrument with an axis of capped trocar cannula assembly 730 and prevent flexing or bending of surgical instrument tip 740 such that surgical instrument tip 740 can be inserted through capped trocar cannula assembly 730 for access to a surgical site without becoming kinked, tangled, and/or damaged. In some embodiments, surgical instrument tip stiffener 750 may slide over surgical instrument tip 740. In many embodiments, one or more portions of capped trocar cannula assembly 730 may prevent at least a portion of surgical instrument tip stiffener 750 from passing through capped trocar cannula assembly 730 with surgical instrument tip 740. In many such embodiments, surgical instrument tip 740 may slide through surgical instrument tip stiffener 750. Embodiments are not limited in this context.

The following examples pertain to further embodiments, from which numerous permutations and configurations will be apparent.

Example 1 is apparatus for aligning a surgical instrument tip with a trocar cannula for providing the surgical instrument tip with access to an interior of an eye, the apparatus comprising: a base; a trocar cannula coupler configured for connecting the apparatus to the trocar cannula along an entry axis of the trocar cannula; and a guidance port having a proximal end and a distal end, the guidance port configured to receive the surgical instrument tip at the proximal end of the guidance port and to guide alignment of the surgical instrument tip with the entry axis of the trocar cannula when the surgical instrument tip is advanced through the guidance port to the distal end of the guidance port.

Example 2 includes the subject matter of Example 1, wherein the guidance port comprises a tapered surface that decreases in diameter as a distance to the distal end of the guidance port decreases to guide alignment of the surgical instrument tip with the entry axis.

Example 3 includes the subject matter of Example 1, wherein the guidance port comprises a concave surface exposed to the ambient pressure, the concave surface configured to guide axial alignment of the surgical instrument tip with the trocar cannula.

Example 4 includes the subject matter of Example 1, wherein the guidance port comprises a cylindric port in axial alignment with the trocar cannula to guide alignment of the surgical instrument tip with the entry axis.

Example 5 includes the subject matter of Example 1, wherein the surgical instrument tip comprises a flexible tip.

Example 6 includes the subject matter of Example 1, wherein the trocar cannula or the trocar cannula coupler comprises at least one leg and wherein the other of the trocar cannula or the trocar cannula coupler comprises a notch to receive the at least one leg for coupling the trocar cannula to the trocar cannula coupler.

Example 7 includes the subject matter of Example 1, wherein the guidance port comprises latex or silicon.

Example 8 includes the subject matter of Example 1, comprising a stiffener slid over the surgical instrument tip.

Example 9 includes the subject matter of Example 1, wherein the guidance port comprises a funnel shaped to guide axial alignment of the surgical instrument tip with the trocar cannula.

Example 10 is a method of configuring a trocar cannula for aligning a surgical instrument tip with an entry axis of a trocar cannula for providing the surgical instrument tip with access to an interior of an eye, the method comprising: attaching a device to the trocar cannula, wherein the device comprises a guidance port having a proximal end and a distal end, the guidance port configured to receive the surgical instrument tip at the proximal end of the guidance port and to guide alignment of the surgical instrument tip with the entry axis of the trocar cannula when the surgical instrument tip is advanced through the guidance port to the distal end of the guidance port.

Example 11 includes the subject matter of Example 10, wherein the guidance port has a first diameter at its proximal end and a second diameter at its distal end, wherein the first diameter is larger than the second diameter.

Example 12 is a surgical instrument and a device for stiffening a tip of the surgical instrument for inserting the surgical instrument tip through a lumen of a trocar cannula, comprising: a surgical instrument having a flexible tip; a surgical instrument tip stiffener comprising a first cylindrical structure with a first outside diameter at a proximal end, a second outside diameter at a distal end, and an inside diameter along an entry axis between the proximal and distal ends, wherein the first outside diameter is larger than the second outside diameter and the second outside diameter is less than or equal to an inside diameter of the lumen of the trocar cannula; wherein the surgical instrument tip stiffener is placed over the flexible tip of the surgical instrument to prevent bending of the surgical instrument tip when the surgical instrument tip is inserted into the trocar cannula.

Example 13 includes the subject matter of Example 12, wherein the first cylindrical structure comprises a rigid cylindrical structure and the cylindrical structure of the surgical instrument tip comprises a flexible cylindrical structure.

Example 14 includes the subject matter of Example 12, wherein the first cylindrical structure comprises a rigid first portion proximate the distal end and a flexible second portion proximate the proximal end.

Example 15 includes the subject matter of Example 14, wherein the flexible second portion comprises a tube.

Example 16 includes the subject matter of Example 12, wherein the surgical instrument tip stiffener comprises a stepped transition from the first outside diameter to the second outside diameter.

Example 17 includes the subject matter of Example 12, wherein the surgical instrument tip stiffener comprises a sloped transition from the first outside diameter to the second outside diameter.

Example 18 includes the subject matter of Example 12, wherein at least a portion of the distal end of the surgical instrument tip stiffener passes through the lumen of the trocar cannula during insertion of the surgical instrument tip through the trocar cannula.

Example 19 includes the subject matter of Example 12, wherein the first outside diameter at the proximal end comprises at least a portion of a flange.

Example 20 includes the subject matter of Example 12, wherein the trocar cannula comprises a valved trocar cannula.

The foregoing description of example embodiments has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner, and may generally include any set of one or more limitations as variously disclosed or otherwise demonstrated herein.

1. An apparatus for aligning a surgical instrument tip with a trocar cannula for providing the surgical instrument tip with access to an interior of an eye, the apparatus comprising:
a base;
a trocar cannula coupler configured for connecting the apparatus to the trocar cannula along an entry axis of the trocar cannula; and
a guidance port having a proximal end and a distal end, the guidance port configured to receive the surgical instrument tip at the proximal end of the guidance port and to guide alignment of the surgical instrument tip with the entry axis of the trocar cannula when the surgical instrument tip is advanced through the guidance port to the distal end of the guidance port.

2. The apparatus of claim 1, wherein the guidance port comprises a tapered surface that decreases in diameter as a distance to the distal end of the guidance port decreases to guide alignment of the surgical instrument tip with the entry axis.

3. The apparatus of claim 1, wherein the guidance port comprises a concave surface exposed to the ambient pressure, the concave surface configured to guide axial alignment of the surgical instrument tip with the trocar cannula.

4. The apparatus of claim 1, wherein the guidance port comprises a cylindric port in axial alignment with the trocar cannula to guide alignment of the surgical instrument tip with the entry axis.

5. The apparatus of claim 1, wherein the surgical instrument tip comprises a flexible tip.

6. The apparatus of claim 1, wherein the trocar cannula or the trocar cannula coupler comprises at least one leg and wherein the other of the trocar cannula or the trocar cannula coupler comprises a notch to receive the at least one leg for coupling the trocar cannula to the trocar cannula coupler.

7. The apparatus of claim 1, wherein the guidance port comprises latex or silicon.

8. The apparatus of claim 1, comprising a stiffener slid over the surgical instrument tip.

9. The apparatus of claim 1, wherein the guidance port comprises a funnel shaped to guide axial alignment of the surgical instrument tip with the trocar cannula.

10. A method of configuring a trocar cannula for aligning a surgical instrument tip with an entry axis of a trocar cannula for providing the surgical instrument tip with access to an interior of an eye, the method comprising:
attaching a device to the trocar cannula, wherein the device comprises a guidance port having a proximal end and a distal end, the guidance port configured to receive the surgical instrument tip at the proximal end of the guidance port and to guide alignment of the surgical instrument tip with the entry axis of the trocar cannula when the surgical instrument tip is advanced through the guidance port to the distal end of the guidance port.

11. The method of claim 10, wherein the guidance port has a first diameter at its proximal end and a second diameter at its distal end, wherein the first diameter is larger than the second diameter.

12. A surgical instrument and a device for stiffening a tip of the surgical instrument for inserting the surgical instrument tip through a lumen of a trocar cannula, comprising:
a surgical instrument having a flexible tip;
a surgical instrument tip stiffener comprising a first cylindrical structure with a first outside diameter at a proximal end, a second outside diameter at a distal end, and an inside diameter along an entry axis between the proximal and distal ends, wherein the first outside diameter is larger than the second outside diameter and the second outside diameter is less than or equal to an inside diameter of the lumen of the trocar cannula;
wherein the surgical instrument tip stiffener is placed over the flexible tip of the surgical instrument to prevent bending of the surgical instrument tip when the surgical instrument tip is inserted into the trocar cannula.

13. The surgical instrument and device of claim 12, wherein the first cylindrical structure comprises a rigid cylindrical structure and the cylindrical structure of the surgical instrument tip comprises a flexible cylindrical structure.

14. The surgical instrument and device of claim 12, wherein the first cylindrical structure comprises a rigid first portion proximate the distal end and a flexible second portion proximate the proximal end.

15. The surgical instrument and device of claim 14, wherein the flexible second portion comprises a tube.

16. The surgical instrument and device of claim 12, wherein the surgical instrument tip stiffener comprises a stepped transition from the first outside diameter to the second outside diameter.

17. The surgical instrument and device of claim 12, wherein the surgical instrument tip stiffener comprises a sloped transition from the first outside diameter to the second outside diameter.

18. The surgical instrument and device of claim 12, wherein at least a portion of the distal end of the surgical instrument tip stiffener passes through the lumen of the trocar cannula during insertion of the surgical instrument tip through the trocar cannula.

19. The surgical instrument and device of claim 12, wherein the first outside diameter at the proximal end comprises at least a portion of a flange.

20. The surgical instrument and device of claim 12, wherein the trocar cannula comprises a valved trocar cannula.
